(19) United States
(12) Patent Application Publication
(10) Pub. No.: US 2025/0040994 A1
(43) Pub. Date: Feb. 6, 2025

(54) DOUBLE-SIDED ULTRASOUND IMAGE ACQUISITION

(71) Applicant: Brainlab AG, Munich (DE)

(72) Inventors: Sebastian STOPP, Munich (DE); Nils FRIELINGHAUS, Munich (DE)

(21) Appl. No.: 18/718,538

(22) PCT Filed: Jan. 18, 2022
(86) PCT No.: PCT/EP2022/051026

§ 371 Date: Jun. 11, 2024

(51) Int. Cl. A61B 34/20; A61B 8/00; A61B 8/08; A61B 8/12

(52) U.S. Cl. CPC A61B 34/20; A61B 8/0841; A61B 8/12; A61B 8/4477; A61B 8/4488; A61B 8/483; A61B 2034/2063

(57) ABSTRACT

The present disclosure relates to an approach of determining the spatial position of ultrasound images by utilizing an ultrasound probe being capable of acquiring three-dimensional ultrasound images in different, particularly in opposite directions. The present disclosure further relates to such an ultrasound probe, a computer program causing a computer to carry out the inventive approach, and a medical system that includes such computer and such ultrasound probe.

### FIELD OF THE INVENTION

The present invention relates to a computer-implemented method of determining the spatial position of ultrasound images, a corresponding computer program, a computer-readable storage medium storing such a program and a computer executing the program, as well as a medical system comprising an electronic data storage device and the aforementioned computer.

### TECHNICAL BACKGROUND

In recent years, ultrasound imaging technology has advanced to a state where it is possible to fabricate ultrasound probes which are small enough for being inserted into body cavities. The images generated via those ultrasound probes provide valuable information as to target and risk organs. However, determining the spatial relation of such image information to co-ordinate systems assigned to external tracking systems has shown very difficult as many tracking modalities are not suited for tracking within body cavities, or regularly come along with significant drawbacks. For example, it is crucial for optical tracking systems to have an uninterrupted line of sight between tracking markers attached to objects to be tracked, and optical sensors or cameras for detecting those tracking markers. Further, EM-tracking systems are highly sensitive to magnetic fields, such that metal objects within or close to the monitored volume of interest may disturb or even prevent accurate tracking.

The present invention has the object of determining the precise spatial position of ultrasound-images obtained via ultrasound probes disposed within body cavities.

The present invention can be used for any computer-assisted procedures which involve the use of ultrasound probes for image acquisition, e.g. in connection with a medical tracking system such as Kick® and Curve®, both products of Brainlab AG.

Aspects of the present invention, examples and exemplary steps and their embodiments are disclosed in the following. Different exemplary features of the invention can be combined in accordance with the invention wherever technically expedient and feasible.

### Exemplary Short Description of the Invention

In the following, a short description of the specific features of the present invention is given which shall not be understood to limit the invention only to the features or a combination of the features described in this section.

The approach suggested herein includes the acquisition of multiple ultrasound images in different directions, i.e. having different fields of view, via the same ultrasound imaging device, wherein at least one first ultrasound image is to provide information as to an object of interest, and wherein at least one second ultrasound image is to register the imaging devices' spatial position with respect to a tracking system, such that the spatial position of the first ultrasound image as well as of objects of interest depicted therein can eventually be determined with respect to the tracking system's co-ordinate system.

### General Description of the Invention

In this section, a description of the general features of the present invention is given for example by referring to possible embodiments of the invention.

In general, the invention reaches the aforementioned object by providing, in a first aspect, a computer-implemented medical method of determining the spatial position of ultrasound images. The method comprises executing, on at least one processor of at least one computer (for example at least one computer being part of a navigation system), the following exemplary steps which are executed by the at least one processor.

In a (for example first) exemplary step, target image data is acquired which describes the spatial relative position between a first three-dimensional ultrasound image showing a target structure, and an imaging device generating the first three-dimensional ultrasound image in a first direction of acquisition.

In a (for example second) exemplary step reference structure data is acquired which describes the spatial position of a reference structure, particularly of a bony reference structure, shown in a second three-dimensional ultrasound image generated via the imaging device in a second direction of acquisition, wherein the second direction of acquisition differs from the first direction of acquisition, particularly wherein the second direction of acquisition is essentially opposite to the first direction of acquisition.

In a (for example third) exemplary step, image position data is determined based on the target image data and the reference structure data, which describes the spatial position of the first three-dimensional ultrasound image.

In other words, a first ultrasound image is acquired which shows the structure of interest, the position of which is to be determined. This may be done via a first ultrasound transducer array of the ultrasound imaging device, which is oriented in the first direction of acquisition and adapted to acquire a three-dimensional ultrasound image. In the same manner, a second three-dimensional ultrasound image is acquired via the ultrasound imaging device or probe in a second direction of acquisition so as to obtain an image of a reference structure the spatial position of which is determined for registration purposes and via means as described further below.

As both of the first transducer array and the second transducer array are calibrated, the spatial position of any structure visible in the acquired ultrasound images can be determined with respect to the ultrasound imaging device, i.e. within a co-ordinate system assigned thereto. Thus, as soon as the absolute spatial position of the reference structure is known, the absolute spatial position of the target structure shown in the first 3D ultrasound image can be calculated based on the absolute spatial position of the reference structure shown in the second three-dimensional ultrasound image.

Assuming that the structures do not move with respect to each other, a single set of ultrasound images would be sufficient for doing so. In reality, however, those structures may move with respect to each other. Thus, the images need to be acquired in regular time intervals so as to “track” the current spatial position of the target structure over time.

The three-dimensional ultrasound images may be acquired in substantially opposite directions, such that the ultrasound imaging device is ideally disposed between the target structure and the reference structure, with the respective transducer arrays facing towards the target structure and the reference structure, respectively.

Acquiring reference structure data may involve determining the spatial position of at least one medical tracking marker being disposed in a known spatial position relative to the reference structure, wherein the spatial position of the at least one medical tracking marker is determined via a medical tracking system, particularly an optical medical tracking system.

In order to acquire the spatial position of the reference structure, any known approach known in the medical environment may be applicable. For example, the reference structure may be palpated with a pointer instrument which is spatially tracked via a medical tracking system. On the other hand, one or more medical tracking markers, for example EM-tracking markers or optical tracking markers may be adhered to the patient's skin, for example adjacent to bony structures such as one or more ribs underneath the skin, which serve as reference structure. With the distance between the tracking markers and the reference structure being small enough, it can be assumed that the tracking marker maintains its position with respect to the reference structure with sufficient accuracy. Thus, once the reference structure has been registered with respect to the one or more tracking markers, its spatial position can be tracked over time by monitoring the spatial position of the one or more tracking markers.

With the relative positions between a) the tracking marker(s) and the reference structure, b) the reference structure and the ultrasound imaging device, and c) the ultrasound imaging device and the target structure being known, the absolute spatial position of the target structure within the patient's body can then be determined via a conventional, external tracking system that may even require a direct line-of-sight for monitoring structures or objects.

Another approach for determining the spatial position of the reference structure within the co-ordinate system of an external tracking system involves acquiring a three-dimensional image dataset that also shows the reference structure and has a known spatial position, for example within a co-ordinate system assigned to the tracking system. Matching this three-dimensional image dataset to the second three-dimensional ultrasound image provided by the ultrasound imaging device provides the required spatial relationship between the external tracking system and the ultrasound imaging device.

For example, such additional three-dimensional image dataset may be acquired via an ultrasound imaging modality that involves the use of an ultrasound probe which is spatially tracked via a medical tracking system, particularly an optical medical tracking system. For example, a hand-held ultrasound probe may be provided with tracking markers which can be recognized by the medical tracking system. With the ultrasound probe being calibrated and the relative position between the generated ultrasound image and the tracking markers being therefore known, the spatial position of the reference structure seen on the additional ultrasound image can therefore be determined within the co-ordinate system of the tracking system. On that basis, the spatial position of the target structure can be determined as described above.

Further, the additional three-dimensional image dataset can also be acquired via any conceivable imaging modality which provides the spatial position of the acquired image content within a global co-ordinate system, for example the co-ordinate system of a medical tracking system. In a more specific example, a spatially tracked fluoroscopy device may be utilized. A fluoroscopic image dataset may not only provide real-time moving images of the patient's anatomy and therefore also of the reference structure, but also provides information as to the spatial position (spatial location and/or spatial orientation) of the structures seen on the images. Again, matching those images to the second three-dimensional ultrasound images provided by the ultrasound imaging device ultimately allows for determining and tracking the spatial position of the target structure within the co-ordinate system assigned to the medical tracking system.

In further examples of the method described herein
acquiring reference structure data may involve the use of at least one spatially tracked ultrasound receiver being adapted to receive ultrasound waves emitted from the imaging device, particularly in the second direction of acquisition, wherein the second three-dimensional ultrasound image is generated based on characteristics of ultrasound waves emitted from the imaging device and received at the at least one ultrasound receiver; or
acquiring reference structure data may involve the use of at least one spatially tracked ultrasound emitter being adapted to emit ultrasound waves received at the imaging device, particularly in the first direction of acquisition, wherein the second three-dimensional ultrasound image is generated based on characteristics of ultrasound waves received at the imaging device and emitted from the at least one ultrasound emitter.

In other words, the ultrasound imaging device forms part of an ultrasound imaging system including multiple components that emit and/or receive ultrasound waves. While the ultrasound imaging device may be disposed in the patient's anatomy, the one or more additional ultrasound receivers and/or the one or more ultrasound emitters are disposed on the outside of the patient's anatomy, and are for example adhered to the patient's skin so as to be tracked via a conventional, external medical tracking system. The relative position between the ultrasound imaging device and the one or more additional ultrasound receivers/ultrasound emitters may for example be determined based on the characteristics of ultrasound waves received at one or more receiving units that were initially emitted with a known characteristic by the one or more emitting units. For example, if an emitting unit such as the ultrasound imaging device is controlled to emit ultrasound waves having a plane wavefront, and these ultrasound waves are received at an angle at a plane array of ultrasound transducers of a receiving unit such as an externally adhered ultrasound receiver, the relative orientation of the emitting ultrasound imaging device and the receiving ultrasound receiver, particularly the orientation of plane ultrasound transducer arrays thereof can be calculated from the time period between the plane wavefront is received first and is received last at the ultrasound receiver, respectively. Additionally, the spatial location and the spatial orientation of the at least one external unit is determined via the medical tracking system. Thus, providing three external units which are concurrently tracked by the medical tracking system allows for triangulating the spatial position of the ultrasound imaging device within the patient's anatomy by calculating at which angles a plane wavefront emitted by the ultrasound imaging device is received at the respective external units. Of course, any other known and applicable approaches for evaluating the characteristics of ultrasound waves may be utilized as a basis for determining the relative position between the ultrasound images and the respective external units. Further, the approach described above also works for a set-up in which the ultrasound imaging device is adapted to receive ultrasound waves which are initially emitted by one or more tracked external units.

With the spatial position of the target structure being determined within the co-ordinate system assigned to the medical tracking system, the positional information may be further processed by a medical navigation system. For example, the three-dimensional ultrasound image showing the target structure may be registered and displayed superimposed with a previously acquired three-dimensional dataset of the patient, for example a MRI-dataset or a CT-image dataset. Moreover, as the medical tracking system is also capable of tracking medical instruments or medical appliances, the ultrasound image showing the target structure can be shown on a display in correct alignment with the tracked medical instruments and medical appliances so as to assist personnel during a medical intervention.

Further, any properties of the respective ultrasound images acquired with the ultrasound imaging device may be selectively chosen so as to best serve the purpose of the respective ultrasound images. For example, the ultrasound image which is to show one or more reference structures may have a large field-of-view so as to cover many reference structures suitable for registering the ultrasound image with other image datasets.

On the other hand, the ultrasound image which is to show the target structure may have, as compared to the ultrasound image showing the reference structure, a high resolution so as to depict even small details of the target structure.

Moreover, for saving the resources of a small and easy to handle ultrasound imaging device or ultrasound probe, the respective ultrasound images may be acquired at different frequencies, and/or in an alternating manner. In cases for which the ultrasound imaging device or ultrasound probe is expected not to move or is expected to only rarely move with respect to the at least one reference structure, the frequency at which the ultrasound images showing the reference structure are acquired may be reduced so as to save transmission capacities which can at the same time be used for transmitting ultrasound images of the target structure at a higher frequency so as to obtain a more fluent depiction of the target structure. Acquiring the respective ultrasound images in an alternating manner also saves resources as there is no need for a simultaneous transmission. With these measures, the ultrasound imaging device or ultrasound probe provided for performing the method described herein can be used more efficiently. This also allows for ultrasound imaging devices or ultrasound probes which are smaller and easier to handle.

In a second aspect, the invention relates to an ultrasound imaging device for acquiring three-dimensional ultrasound images, which comprises
a first transducer array including a plurality of ultrasound transducers having a first direction of acquisition; and
a second transducer array including a plurality of ultrasound transducers having a second direction of acquisition differing from the first direction of acquisition, particularly wherein the second direction of acquisition is oriented essentially opposite to the first direction of acquisition.

It should be noted here that any ultrasound imaging device or any ultrasound probe described in more detail in the following lines may be utilised for carrying out any of the methods described in the lines above.

Basically, an ultrasound probe for carrying out the inventive approach comprises two or more transducer arrays which face in different directions so as to acquire three-dimensional ultrasound images with different fields-of-view. In a specific example, the probe includes two transducer arrays facing in opposite directions for acquiring ultrasound images of a target structure and of a reference structure, respectively.

For serving specific requirements, the ultrasound probe may have one more of the following properties:
the ultrasound imaging device comprises a flat design body having a width, a depth and a height, wherein the width and the depth of the body are substantially larger than the height of the body, wherein the first transducer array and the second transducer array are disposed at the large sides of the body facing in substantially opposite directions;
the ultrasound imaging device comprises a single acoustic absorber layer being sandwiched between the first transducer array and the second transducer array;
at least one of the first transducer array and the second transducer array features an acoustic lens, particularly wherein the curvature of an acoustic lens of the first transducer array and the curvature of an acoustic lens of the second transducer array differ from each other;
the plurality of transducer elements of at least one of the first transducer array and the second transducer array are controlled to emit ultrasound waves having a curved wavefront, particularly wherein the curvature of the wavefront emitted via the first transducer array and the curvature of the wavefront emitted via the second transducer array differ from each other;
the plurality of transducer elements of at least one of the first transducer array and the second transducer array are disposed in an n×m-matrix or an n×n-matrix, and/or are controlled via an RCA (Row-Column-Addressing)-scheme;
the first transducer array provides ultrasound images of a different, particularly of a higher resolution than the second transducer array;
the first transducer array and the second transducer array share the same wireless connection or cable connection for transmitting data for controlling the first transducer array and the second transducer array an/or for transmitting data describing said three-dimensional ultrasound images;
the first transducer array and the second transducer array are controlled to acquire ultrasound images in an alternating manner;
the first transducer array and the second transducer array are controlled to acquire ultrasound images at different frequencies.

Each one of the transducer arrays may comprise any number of ultrasound transducers arranged in any desired spatial configuration. One or more of the transducer arrays may feature a matrix or two-dimensional arrangement of ultrasound transducer elements, which allows for generating a three-dimensional ultrasound image from ultrasound waves received at the individual transducer elements of the respective two-dimensional array. The transducer elements of such two-dimensional array or matrix may be controlled individually or based on an RCA (row-column-addressing)-scheme as described below.

For generating, processing and/or analysing the signals received from the transducer elements, the ultrasound imaging device or probe may connect, via a wireless or via a cable data connection, to an external unit. This unit may connect to the transducer elements of the probe for controlling the generation of ultrasound waves as well as for processing the signals received therefrom. In particular, such unit may be configured to selectively control predefined transducer elements of at least one of the transducer arrays to convert electrical energy in sound energy and/or to convert sound energy in electrical energy. It is generally conceivable that this signal unit is capable of addressing, i.e. controlling each transducer element individually in a “fully addressed” configuration. An easier and therefore more cost efficient addressing scheme for controlling a two-dimensional array of transducer elements is called RCA (“row column addressing scheme”). With RCA, the transducer elements are not individually controlled. Rather, full rows and/or full columns within the two-dimensional transducer element array or matrix are addressed to send and/or receive ultrasound waves. RCA allows for controlling a plurality of transducer elements to form linear senders and/or receivers within rows and/or columns of a two-dimensional array or matrix of transducer elements. Thus, a two-dimensional array or matrix of transducer elements of at least one transducer array may provide one or more linear senders and/or receivers which may further be oriented in a predefined angle, particularly parallel or perpendicularly with respect to each other.

Further, hybrid transducers may be used in which one transducer material is used for generating ultrasound waves, whereas another material is used for receiving ultrasound waves.

In a third aspect, the invention is directed to a computer program comprising instructions which, when the program is executed by at least one computer, causes the at least one computer to carry out method according to the first aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the first aspect. The signal wave is in one example a data carrier signal carrying the aforementioned computer program. A computer program stored on a disc is a data file, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the second aspect therefore may alternatively or additionally relate to a data stream representative of the aforementioned program, i.e. comprising the program.

In a fourth aspect, the invention is directed to a computer-readable storage medium on which the program according to the second aspect is stored. The program storage medium is for example non-transitory.

In a fifth aspect, the invention is directed to at least one computer (for example, a computer), comprising at least one processor (for example, a processor), wherein the program according to the second aspect is executed by the processor, or wherein the at least one computer comprises the computer-readable storage medium according to the third aspect.

In a sixth aspect, the invention is directed to a medical system, comprising:
a) the at least one computer according to the fifth aspect;
b) the at least one ultrasound imaging device according to the second aspect wherein the at least one computer is operably coupled to the ultrasound imaging device for issuing a control signal to the ultrasound imaging device for controlling the operation of the ultrasound imaging device, and for receiving three-dimensional ultrasound images to determine the spatial position of at least one of the three-dimensional ultrasound images.

Alternatively or additionally, the invention according to the sixth aspect is directed to a for example non-transitory computer-readable program storage medium storing a program for causing the computer according to the fifth aspect to execute the data processing steps of the method according to the first aspect.

For example, the invention does not involve or in particular comprise or encompass an invasive step which would represent a substantial physical interference with the body requiring professional medical expertise to be carried out and entailing a substantial health risk even when carried out with the required professional care and expertise.

More particularly, the invention does not involve or in particular comprise or encompass any surgical or therapeutic activity. For this reason alone, no surgical or therapeutic activity and in particular no surgical or therapeutic step is necessitated or implied by carrying out the invention.

### Definitions

In this section, definitions for specific terminology used in this disclosure are offered which also form part of the present disclosure.

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

The n-dimensional image of a body is registered when the spatial location of each point of an actual object within a space, for example a body part in an operating theatre, is assigned an image data point of an image (CT, MR, etc.) stored in a navigation system.

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

The movements of the treatment body parts are for example due to movements which are referred to in the following as “vital movements”. Reference is also made in this respect to EP 2 189 943 A1 and EP 2 189 940 A1, also published as US 2010/0125195 A1 and US 2010/0160836 A1, respectively, which discuss these vital movements in detail. In order to determine the position of the treatment body parts, analytical devices such as x-ray devices, CT devices or MRT devices are used to generate analytical images (such as x-ray images or MRT images) of the body. For example, analytical devices are constituted to perform medical imaging methods. Analytical devices for example use medical imaging methods and are for example devices for analysing a patient's body, for instance by using waves and/or radiation and/or energy beams, for example electromagnetic waves and/or radiation, ultrasound waves and/or particles beams. Analytical devices are for example devices which generate images (for example, two-dimensional or three-dimensional images) of the patient's body (and for example of internal structures and/or anatomical parts of the patient's body) by analysing the body. Analytical devices are for example used in medical diagnosis, for example in radiology. However, it can be difficult to identify the treatment body part within the analytical image. It can for example be easier to identify an indicator body part which correlates with changes in the position of the treatment body part and for example the movement of the treatment body part. Tracking an indicator body part thus allows a movement of the treatment body part to be tracked on the basis of a known correlation between the changes in the position (for example the movements) of the indicator body part and the changes in the position (for example the movements) of the treatment body part. As an alternative to or in addition to tracking indicator body parts, marker devices (which can be used as an indicator and thus referred to as “marker indicators”) can be tracked using marker detection devices. The position of the marker indicators has a known (predetermined) correlation with (for example, a fixed relative position relative to) the position of indicator structures (such as the thoracic wall, for example true ribs or false ribs, or the diaphragm or intestinal walls, etc.) which for example change their position due to vital movements.

Elastic fusion transformations (for example, elastic image fusion transformations) are for example designed to enable a seamless transition from one dataset (for example a first dataset such as for example a first image) to another dataset (for example a second dataset such as for example a second image). The transformation is for example designed such that one of the first and second datasets (images) is deformed, for example in such a way that corresponding structures (for example, corresponding image elements) are arranged at the same position as in the other of the first and second images. The deformed (transformed) image which is transformed from one of the first and second images is for example as similar as possible to the other of the first and second images. Preferably, (numerical) optimisation algorithms are applied in order to find the transformation which results in an optimum degree of similarity. The degree of similarity is preferably measured by way of a measure of similarity (also referred to in the following as a “similarity measure”). The parameters of the optimisation algorithm are for example vectors of a deformation field. These vectors are determined by the optimisation algorithm in such a way as to result in an optimum degree of similarity. Thus, the optimum degree of similarity represents a condition, for example a constraint, for the optimisation algorithm. The bases of the vectors lie for example at voxel positions of one of the first and second images which is to be transformed, and the tips of the vectors lie at the corresponding voxel positions in the transformed image. A plurality of these vectors is preferably provided, for instance more than twenty or a hundred or a thousand or ten thousand, etc. Preferably, there are (other) constraints on the transformation (deformation), for example in order to avoid pathological deformations (for instance, all the voxels being shifted to the same position by the transformation). These constraints include for example the constraint that the transformation is regular, which for example means that a Jacobian determinant calculated from a matrix of the deformation field (for example, the vector field) is larger than zero, and also the constraint that the transformed (deformed) image is not self-intersecting and for example that the transformed (deformed) image does not comprise faults and/or ruptures. The constraints include for example the constraint that if a regular grid is transformed simultaneously with the image and in a corresponding manner, the grid is not allowed to interfold at any of its locations. The optimising problem is for example solved iteratively, for example by means of an optimisation algorithm which is for example a first-order optimisation algorithm, such as a gradient descent algorithm. Other examples of optimisation algorithms include optimisation algorithms which do not use derivations, such as the downhill simplex algorithm, or algorithms which use higher-order derivatives such as Newton-like algorithms. The optimisation algorithm preferably performs a local optimisation. If there is a plurality of local optima, global algorithms such as simulated annealing or generic algorithms can be used. In the case of linear optimisation problems, the simplex method can for instance be used.

In the steps of the optimisation algorithms, the voxels are for example shifted by a magnitude in a direction such that the degree of similarity is increased. This magnitude is preferably less than a predefined limit, for instance less than one tenth or one hundredth or one thousandth of the diameter of the image, and for example about equal to or less than the distance between neighbouring voxels. Large deformations can be implemented, for example due to a high number of (iteration) steps.

The determined elastic fusion transformation can for example be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can for example be determined on the basis of a determined correlation between the first and second datasets.

A fixed position, which is also referred to as fixed relative position, in this document means that two objects which are in a fixed position have a relative position which does not change unless this change is explicitly and intentionally initiated. A fixed position is in particular given if a force or torque above a predetermined threshold has to be applied in order to change the position. This threshold might be 10 N or 10 Nm. In particular, the position of a sensor device remains fixed relative to a target while the target is registered or two targets are moved relative to each other. A fixed position can for example be achieved by rigidly attaching one object to another. The spatial location, which is a part of the position, can in particular be described just by a distance (between two objects) or just by the direction of a vector (which links two objects). The alignment, which is another part of the position, can in particular be described by just the relative angle of orientation (between the two objects).

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates the basic steps of the method according to the first aspect;

FIG. 2 shows an embodiment of the ultrasound imaging device according to the second aspect;

FIG. 3 shows a cross-sectional view of the ultrasound imaging device of FIG. 2; and

FIGS. 4 to 6 schematically show different embodiments of a medical system according to the sixth aspect.

### DESCRIPTION OF EMBODIMENTS

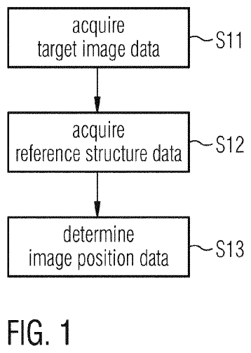

FIG. 1 illustrates the basic steps of the method according to the first aspect, in which the step S11 encompasses acquiring target image data, step S12 encompasses acquiring reference structure data and step S13 encompasses determining image position data based on the target image data and the reference structure data.

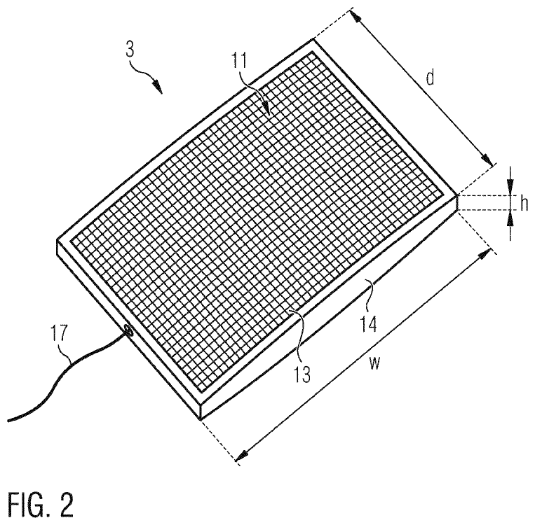

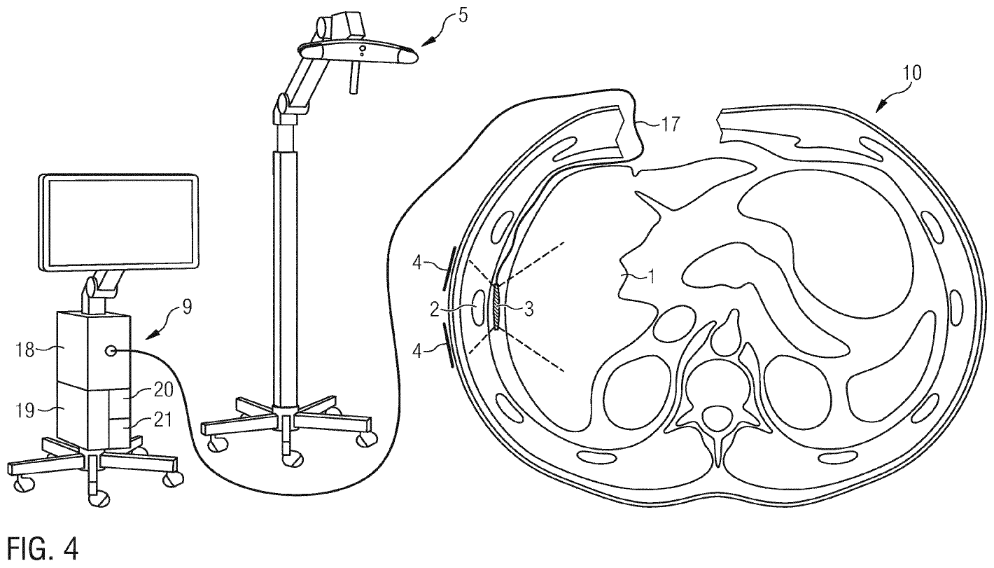

FIG. 2 shows an embodiment of the ultrasound probe according to the second aspect. The probe 3 features a flat, rectangular body 14 having a height h which is substantially smaller than the width w and the depth d of the body 14. Having a flat panel form factor, the probe 3 can be easily introduced into a patient's anatomy (cf. FIGS. 4 to 6) and disposed of a location in-between structures which are to be shown in three-dimensional ultrasound images. For this purpose, probe 3 features an array 11, 12 including a plurality of ultrasound transducer elements 13 on each one of its large opposite surfaces, only one of which is shown in FIG. 2. Both of the transducer arrays 11 and 12 are controlled via a joint cable connection that connects to a beam forming device 18 (cf. FIGS. 4 to 6) which controls the transducer elements 13 of arrays 11 and 12 to receive and to emit ultrasound waves, for example via an RCA-scheme as described further above. Moreover, the beam forming device 18 also receives via the cable connection 17 signals, as to the ultrasound waves received at the transducer elements 13 of the respective arrays 11 and 12, which are eventually processed to create three-dimensional ultrasound images.

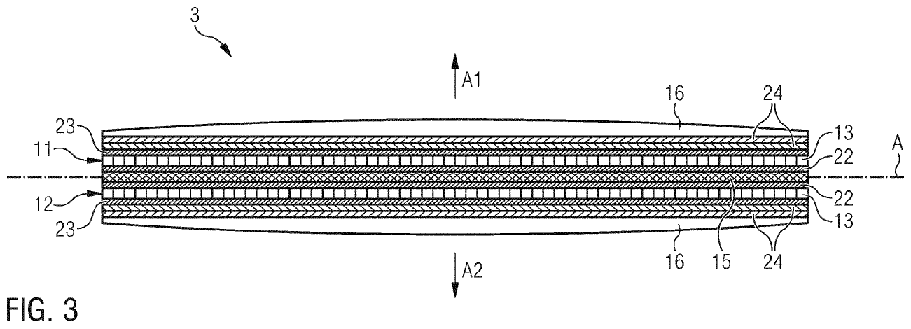

FIG. 3 shows a cross-sectional view of the ultrasound probe 3 of FIG. 2. In the shown embodiment, the transducer array 11 faces upwards in a direction A1 for emitting/receiving ultrasound waves, whereas the transducer array 12 faces in an opposite downward direction A2 for emitting/receiving ultrasound waves. Each one of the arrays 11 and 12 is flanked by ground electrodes 22 and signal electrodes 23 for controlling the individual transducer elements 13 and/or for transmitting signals as to ultrasound waves received at the individual transducer elements 13. Both of the surfaces of the probe 3 are covered by an acoustic lens 16 for creating a desired curvature of the wavefront of the ultrasound waves emitted by the respective transducer arrays 11 and 12 in the upward direction A1 and the downward direction A2, respectively. Both of the acoustic lenses 16 are coupled to the respective transducer arrays 11 and 12 via acoustic matching layers 24 in a manner known in the art.

At the central plane A of the probe 3, the upward facing transducer array 11 and the downward facing transducer array 12 share the same acoustic absorber layer 15 which avoids redundancies in the probe 3 components and therefore allows for an even sleeker cross-sectional footprint of the probe 3.

FIGS. 4 to 6 show set-ups for determining the spatial position of an ultrasound image showing an anatomical structure.

In the example shown in FIG. 4, the ultrasound probe 3 of FIG. 2 is disposed in the patient's body 10 such that one of its ultrasound transducer arrays faces towards the patient's liver 1 for acquiring a three-dimensional ultrasound image thereof. Thus, probe 3 is placed between the liver 1 and one or more of the patient's ribs 2 such that its opposite transducer arrays faces towards the one or more ribs 2. As shown in FIG. 2, the outward-facing transducer array 12 features a wider field of view than the inward-facing transducer array 11, which may be accomplished with acoustic lenses 16 (cf. FIG. 3) having a higher convex curvature on the outward-facing side of the probe 3. On the other hand, the inward-facing transducer array 11 provides ultrasound images having a higher resolution as the given number of transducer elements only needs to cover a comparatively smaller field of view.

With the probe 3 being calibrated, any structures seen in the inward facing image or the outward facing image can be positionally determined with respect to probe 3 and therefore also with respect to each other.

On the outward facing side of probe 3, optical tracking markers 4 are adhered to the patient's skin in a known spatial relationship with respect to a patient's rib 2, which can be accomplished via a known registration procedure. With the tracking markers 4 being spatially tracked via a stereoscopic camera array of a tracking system 5 and assuming that the relative position between tracking markers 4 and rib 2 remains invariant, the spatial position of rib 2 can be calculated within the co-ordinate system of the tracking system 5. The position of rib 2 is therefore not only known within the co-ordinate system of tracking system 5, but also within the co-ordinate system of the probe 3. The spatial position of any structures within the inward-facing field of view of probe 3 can then be calculated within the co-ordinate system assigned to tracking system 5 and further processed for computer-assisted-surgery by a computer 19 of the navigation system 9, which includes a processor 20 and a program storage medium 21. A beam-forming-device 18 connects to probe 3 via a cable connection 17 for controlling the plurality of transducer elements 13 of the respective transducer arrays 11 and 12 to emit and/or receive ultrasound waves, and for transmitting signals describing ultrasound waves which were received at the respective transducer arrays 11 and 12 of probe 3.

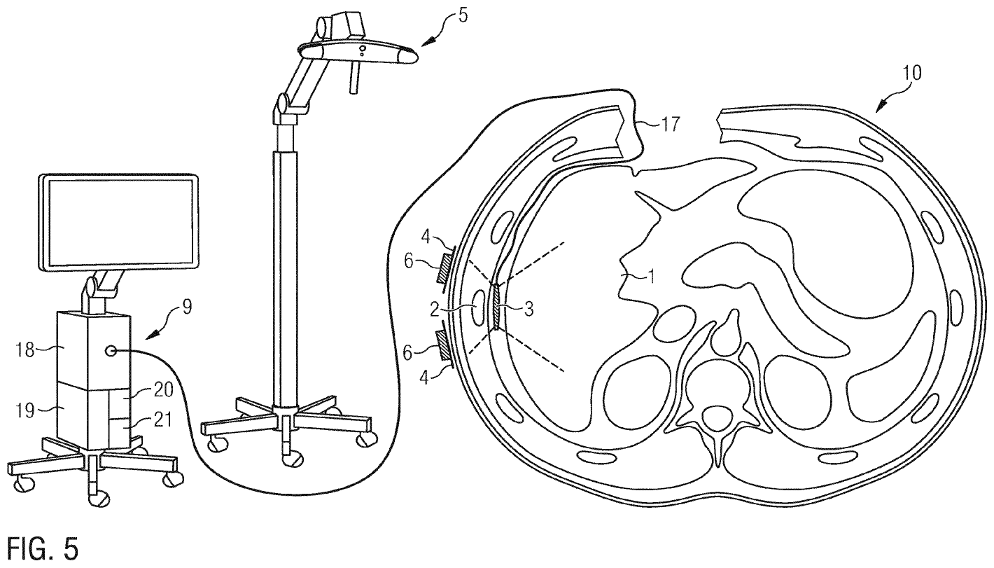

FIG. 5 shows another embodiment of a setup for determining the spatial position of ultrasound images and the image content thereof. The embodiment shown in FIG. 5 distinguishes from the example shown in FIG. 4 in that tracking markers 4 are provided with additional ultrasound probes 6, each of which acquiring a three-dimensional ultrasound image of rib 2. As the external ultrasound probes 6 are spatially tracked by tracking system 5, matching the ultrasound images received from the external ultrasound probes 6 on the one hand and from ultrasound probe 3 on the other hand allows for calculating the spatial position of the inward-facing image and the image content thereof within the co-ordinate system assigned to tracking system 5.

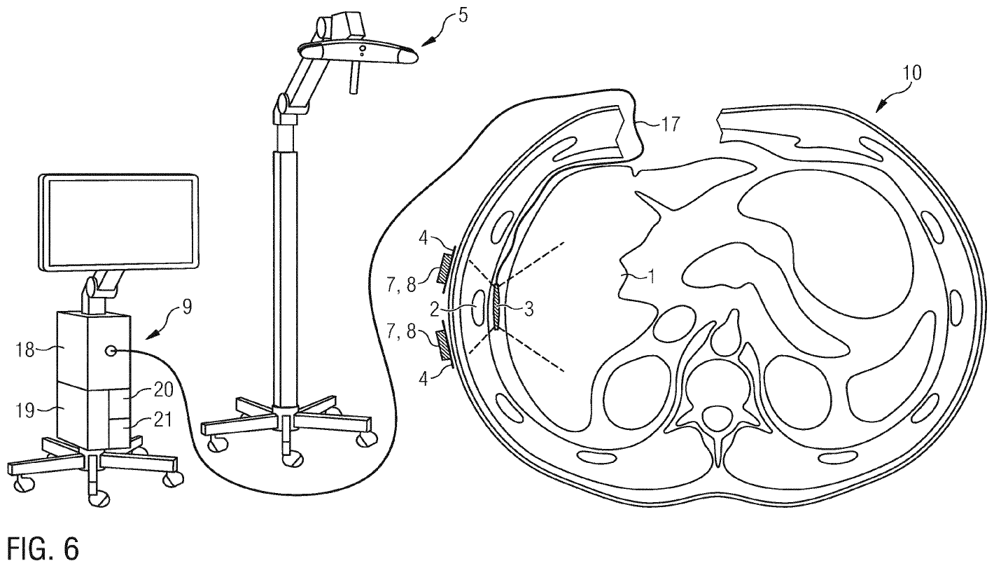

FIG. 6 shows another embodiment of a setup for determining the spatial position of ultrasound images and the image content thereof. The embodiment shown in FIG. 6 distinguishes from the embodiment shown in FIG. 5 in that the external tracking markers 4 are provided with ultrasound receivers 7 and/or ultrasound emitters 8 instead of ultrasound probes 6 which are capable of emitting and receiving ultrasound waves. Thus, receivers 7 and emitters 8 cooperate with ultrasound probe 3 so as to generate outward-facing ultrasound images of rib 2. Consequently, the position of receivers 7 and emitters 8 with respect to the ultrasound probe 3 needs to be determined, for example by evaluating the characteristics of ultrasound waves received at receivers 7, which were initially emitted with known characteristics by ultrasound probe 3, and by eventually triangulating the position of ultrasound probe 3 with respect to a plurality of ultrasound receivers 7.

1. A computer-implemented method of determining the spatial position of ultrasound images, the method comprising:
acquiring target image data that describes a spatial relative position between a first three-dimensional ultrasound image showing a target structure, and an imaging device generating the first three-dimensional ultrasound image in a first direction of acquisition;
acquiring reference structure data that describes the spatial position of a reference structure shown in a second three-dimensional ultrasound image generated via the imaging device in a second direction of acquisition, wherein the second direction of acquisition differs from the first direction of acquisition; and
determining image position data based on the target image data and the reference structure data, wherein the image position data describes the spatial position of the first three-dimensional ultrasound image.

2. The method according to claim 1, wherein the acquiring the reference structure data comprises determining the spatial position of at least one medical tracking marker being disposed in a known spatial position relative to the reference structure, using an optical medical tracking system.

3. The method according to claim 1, wherein the acquiring the reference structure data comprises acquiring a three-dimensional image dataset showing the reference structure and having a known spatial position, and further comprising matching the second three-dimensional ultrasound image to the three-dimensional image dataset.

4. The method according to claim 3, wherein the three-dimensional image dataset is acquired via an ultrasound modality comprising use of an ultrasound probe that is spatially tracked via an associated optical medical tracking system.

5. The method according to claim 3, wherein the acquiring the three-dimensional image dataset comprises acquiring the three-dimensional image dataset via fluoroscopy using a fluoroscopy device.

6. The method according to claim 1, wherein:
the acquiring the reference structure data comprises using at least one spatially tracked ultrasound receiver being adapted to receive ultrasound waves emitted from the imaging device, in the second direction of acquisition, wherein the second three-dimensional ultrasound image is generated based on characteristics of ultrasound waves emitted from the imaging device and received at the at least one ultrasound receiver; or
the acquiring the reference structure data comprises using at least one spatially tracked ultrasound emitter being adapted to emit ultrasound waves received at the imaging device, in the first direction of acquisition, wherein the second three-dimensional ultrasound image is generated based on characteristics of ultrasound waves received at the imaging device and emitted from the at least one ultrasound emitter.

7. The method according to claim 1, wherein:
the reference structure data describes the spatial position of the reference structure in a co-ordinate system assigned to an associated optical medical tracking system; and/or
the image position data describes the spatial position of the first three-dimensional ultrasound image in a co-ordinate system assigned to the associated optical medical tracking system.

8. The method according to claim 1, further comprising transmitting the determined image position data to an associated navigation system for further processing comprising registering the first three-dimensional ultrasound image with a three-dimensional image dataset of a patient and/or for performing Computer-Assisted-Surgery based on the first three-dimensional ultrasound image.

9. The method according to claim 1, wherein the field of view of the second ultrasound image differs from the field of view of the first three-dimensional ultrasound image, wherein the field of view of the second three-dimensional ultrasound image is wider than the field of view of the first three-dimensional ultrasound image.

10. The method according to claim 1, wherein the resolution of the first three-dimensional ultrasound image differs from the resolution of the second three-dimensional ultrasound image, wherein the resolution of the first three-dimensional ultrasound image is higher than the resolution of the second three-dimensional ultrasound image.

11. The method according to claim 1, further comprising acquiring a plurality of first three-dimensional ultrasound images and a plurality of second three-dimensional ultrasound images, wherein:
the plurality of first three-dimensional ultrasound images is acquired at a different frequency than the plurality of second three-dimensional ultrasound images, comprising a higher frequency, and/or
the plurality of first three-dimensional ultrasound images and the plurality of second three-dimensional ultrasound images are acquired in an alternating manner.

12. An ultrasound imaging device operable to acquire three-dimensional ultrasound images, the ultrasound imaging device comprising:
a first transducer array comprising a plurality of ultrasound transducers having a first direction of acquisition;
a second transducer array comprising a plurality of ultrasound transducers having a second direction of acquisition differing from the first direction of acquisition; and
a flat design body having a width, a depth, and a height, wherein the width and the depth of the body are larger than the height of the body, wherein the first and second transducer arrays are disposed at the large sides of the body facing in substantially opposite directions.

13. The ultrasound imaging device according to claim 12, comprising one or more properties of:
the ultrasound imaging device comprises a single acoustic absorber layer being sandwiched between the first transducer array and the second transducer array;
at least one of the first transducer array and the second transducer array features an acoustic lens, wherein the curvature of an acoustic lens of the first transducer array and the curvature of an acoustic lens of the second transducer array differ from each other;
the plurality of transducer elements of at least one of the first transducer array and the second transducer array are controlled to emit ultrasound waves having a curved wavefront, wherein the curvature of the wavefront emitted via the first transducer array and the curvature of the wavefront emitted via the second transducer array differ from each other;
the plurality of transducer elements of at least one of the first transducer array and the second transducer array are disposed in an n*m-matrix or an n*n-matrix, and/or are controlled via a Row-Column-Addressing (RCA) scheme;
the first transducer array provides ultrasound images of a higher resolution than the second transducer array;
the first transducer array and the second transducer array share the same wireless connection or cable connection for transmitting data for controlling the first transducer array and the second transducer array an/or for transmitting data describing said three-dimensional ultrasound images;
the first transducer array and the second transducer array are controlled to acquire ultrasound images in an alternating manner; and/or
the first transducer array and the second transducer array are controlled to acquire ultrasound images at different frequencies.

14. A non-transient computer-readable storage medium storing instructions thereon that, when the instructions are executed by processor of an associated computer cause the computer to perform a method of determining the spatial position of ultrasound images, by:
acquiring target image data that describes a spatial relative position between a first three-dimensional ultrasound image showing a target structure, and an imaging device generating the first three-dimensional ultrasound image in a first direction of acquisition;
acquiring reference structure data that describes the spatial position of a reference structure, shown in a second three-dimensional ultrasound image generated via the imaging device in a second direction of acquisition, wherein the second direction of acquisition differs from the first direction of acquisition; and
determining image position data based on the target image data and the reference structure data, wherein the image position data describes the spatial position of the first three-dimensional ultrasound image.

15. A medical system, comprising:
at least one computer comprising a processor and a non-transient computer-readable storage medium storing instructions thereon that are executable by the processor; and
an ultrasound imaging device operable to acquire three-dimensional ultrasound images, comprising:
an ultrasound imaging device operable to acquire three-dimensional ultrasound images, the ultrasound imaging device comprising:
a first transducer array comprising a plurality of ultrasound transducers having a first direction of acquisition;
a second transducer array comprising a plurality of ultrasound transducers having a second direction of acquisition differing from the first direction of acquisition; and
a flat design body having a width, a depth, and a height, wherein the width and the depth of the body are larger than the height of the body, wherein the first and second transducer arrays are disposed at the large sides of the body facing in substantially opposite directions,
wherein the at least one computer is operably coupled to the ultrasound imaging device for issuing a control signal to the ultrasound imaging device for controlling an operation of the ultrasound imaging device, and for receiving three-dimensional ultrasound images to determine the spatial position of at least one of the three-dimensional ultrasound images.
